(19) United States
(12) Patent Application Publication
(10) Pub. No.: US 2018/0177493 A1
(43) Pub. Date: Jun. 28, 2018

(54) ULTRASONIC MEASUREMENT APPARATUS AND CONTROL METHOD FOR ULTRASONIC MEASUREMENT APPARATUS

(71) Applicant: Seiko Epson Corporation, Tokyo (JP)

(72) Inventors: Masaki HAYASHI, Matsumoto (JP); Ryutaro SEKI, Suwa (JP)

(21) Appl. No.: 15/848,456

(22) Filed: Dec. 20, 2017

(30) Foreign Application Priority Data
Dec. 28, 2016 (JP) 2016-256451

(51) Int. Cl. A61B 8/08; A61B 8/00; G06T 7/13; G06T 7/00

(52) U.S. Cl. CPC A61B 8/5215; A61B 8/4444; A61B 8/54; A61B 8/4483; G06T 2207/10132; A61B 8/467; A61B 8/5207; G06T 7/13; G06T 7/0012; A61B 8/4405

(57) ABSTRACT

An ultrasonic measurement apparatus includes a calculation processing section that performs weight selection of selecting one of a predetermined weight and an adaptive weight, as a signal combination weight for the reception beam forming process, for each processing target point of a target region, and generates an ultrasonic image by performing the reception beam forming process, and the calculation processing section performs edge detection with respect to the target region on the basis of the received signal, extraction of extracting an edge portion satisfying a predetermined condition with respect to a beam direction in the reception beam forming process related to the processing target point among edge portions detected through the edge detection, and weight selection of selecting the predetermined weight as the signal combination weight in a case where the processing target point is the extracted edge portion.

### BACKGROUND

### 1. Technical Field

The present invention relates to an ultrasonic measurement apparatus or the like performing ultrasonic measurement.

### 2. Related Art

In the related art, there is an ultrasonic measurement apparatus which applies ultrasonic beams by using an ultrasonic probe in which a plurality of ultrasonic elements (ultrasonic vibrators) are arranged, and generates an image of a target region inside a living body or a structure in order to use the image for diagnosis or inspection. In image generation, a reception beam forming (BF) process of combining received signals which are received by respective channels (or respective ultrasonic elements) with each other is performed. A resolution of an image is low in a simple reception beam forming process, and thus techniques for obtaining an image with a higher resolution have been developed. For example, one of the techniques is an adaptive beam forming process (adaptive weighted value beam forming technique) disclosed in JP-A-2012-170826.

However, if the adaptive beam forming process is performed, there is a case where it is hard to observe an interface of a living tissue, a structure, or the like in a generated ultrasonic image. If the interface is hardly observed, a position of the living tissue, the structure, or the like may not be recognized at a glance, and this may cause troubles in diagnosis or inspection.

### SUMMARY

An advantage of some aspects of the invention is to provide a technique capable of improving the visibility of an interface in a case where the interface is present in an image diagnosis target region or an image inspection target region.

A first aspect of the invention is directed to an ultrasonic measurement apparatus including a probe that is provided with a plurality of channels receiving ultrasonic waves; and a calculation processing section that performs a calculation process on a received signal which is received by each channel, performs weight selection of selecting one of a predetermined weight and an adaptive weight for performing an adaptive beam forming process as a reception beam forming process, as a signal combination weight for the reception beam forming process, for each processing target point of a target region, and generates an ultrasonic image by performing the reception beam forming process, in which the calculation processing section performs edge detection with respect to the target region on the basis of the received signal, extraction of extracting an edge portion satisfying a predetermined condition with respect to a beam direction in the reception beam forming process related to the processing target point among edge portions detected through the edge detection, and the weight selection of selecting the predetermined weight as the signal combination weight in a case where the processing target point is the extracted edge portion.

As a ninth aspect of the invention, the invention may be configured as a control method for an ultrasonic measurement apparatus including a probe provided with a plurality of channels receiving ultrasonic waves, the control method including performing edge detection with respect to a target region on the basis of a received signal which is received by each of the channels; extracting an edge portion satisfying a predetermined condition with respect to a beam direction in a reception beam forming process related to a processing target point among edge portions detected through the edge detection; and performing weight selection of selecting one of a predetermined weight and an adaptive weight for performing an adaptive beam forming process as a reception beam forming process, as a signal combination weight for the reception beam forming process, for each processing target point of the target region, and performing the reception beam forming process, in which the weight selection includes selecting the predetermined weight as the signal combination weight in a case where the processing target point is the extracted edge portion.

According to the first or ninth aspect of the invention, edge detection can be performed with respect to a target region, and an edge portion satisfying a predetermined condition with respect to a beam direction in a reception BF process related to a processing target point can be extracted. In a case where a processing target point is the extracted edge portion, a reception beam forming process can be performed by using the predetermined weight as the signal combination weight. Therefore, it is possible to improve the visibility of an interface present in the target region in a generated ultrasonic image.

As a second aspect of the invention, the ultrasonic measurement apparatus according to the first aspect of the invention may be configured such that the predetermined condition includes at least a condition in which an extension direction of the edge portion is not orthogonal to the beam direction.

According to the second aspect of the invention, it is possible to extract an edge portion of which an extension direction is not orthogonal to a beam direction. In a case where a processing target point is the extracted edge portion, a reception beam forming process can be performed by using the predetermined weight as the signal combination weight.

As a third aspect of the invention, the ultrasonic measurement apparatus according to the first or second aspect of the invention may be configured such that the probe is a probe capable of performing linear scanning, and the extraction is to extract the edge portion satisfying the predetermined condition by extracting the edge portion of which the extension direction is not parallel to a scanning direction in the linear scanning.

According to the third aspect of the invention, it is possible to extract an edge portion of which an extension direction is not parallel to a scanning direction in linear scanning. In a case where a processing target point is the extracted edge portion, a reception beam forming process can be performed by using the predetermined weight as the signal combination weight.

As a fourth aspect of the invention, the ultrasonic measurement apparatus according to the first or second aspect of the invention may be configured such that the probe is a probe capable of performing linear scanning, and the extraction includes setting the edge portion of which an extension direction is parallel to a scanning direction in the linear scanning as an exclusion target of the extraction, and extracting an edge portion of which an extension direction is not orthogonal to the beam direction and which has a length of a predetermined length or more from edge portions which are not the exclusion target as the edge portion satisfying the predetermined condition.

According to the fourth aspect of the invention, it is possible to extract an edge portion of which an extension direction is not orthogonal to a beam direction and which has a length of a predetermined length or more after setting an edge portion which is parallel to a scanning direction in linear scanning as an extraction exclusion target. In a case where a processing target point is the extracted edge portion, a reception beam forming process can be performed by using the predetermined weight as the signal combination weight.

As a fifth aspect of the invention, the ultrasonic measurement apparatus according to the first or second aspect of the invention may be configured such that the probe is a probe capable of performing sector scanning, the extraction includes extracting the edge portion satisfying the predetermined condition for each beam direction, and the weight selection includes using the predetermined weight as the signal combination weight in a case where the edge portion extracted to correspond to the beam direction is the processing target point.

According to the fifth aspect of the invention, it is possible to extract an edge portion satisfying the predetermined condition for each beam direction. In a case where a processing target point is an edge portion extracted to correspond to the beam direction, a reception beam forming process can be performed by using the predetermined weight as the signal combination weight.

As a sixth aspect of the invention, the ultrasonic measurement apparatus according to the fifth aspect of the invention may be configured such that the predetermined condition includes a condition in which an edge portion has a length of a predetermined length or more, and the calculation processing section sets the predetermined length to be variable depending on a distance to the processing target point along the beam direction.

According to the sixth aspect of the invention, it is possible to extract an edge portion having a length corresponding to a distance to a processing target point along a beam direction.

As a seventh aspect of the invention, the ultrasonic measurement apparatus according to any one of the first to sixth aspects of the invention may be configured such that the predetermined weight is defined depending on a value which the adaptive weight can take.

According to the seventh aspect of the invention, it is possible to perform a reception beam forming process can be performed by using the predetermined weight which is defined depending on a value which the adaptive weight can take, as the signal combination weight.

As an eighth aspect of the invention, the ultrasonic measurement apparatus according to any one of the first to seventh aspects of the invention may be configured such that the adaptive weight is a weight in which a value corresponding to the amplitude of the received signal of each channel is variable, and the adaptive beam forming process is a signal combination process in which weighted addition is performed on the received signal by using the adaptive weight.

According to the eighth aspect of the invention, since the adaptive beam forming process is performed, it is possible to increase a resolution (azimuth resolution) and thus to improve quality of an ultrasonic image.

### BRIEF DESCRIPTION OF THE DRAWINGS

The invention will be described with reference to the accompanying drawings, wherein like numbers reference like elements.

FIG. 1 is a diagram illustrating a system configuration example of an ultrasonic measurement apparatus.

FIG. 2 is a schematic diagram illustrating an example of a scanning range.

FIG. 3 is a diagram illustrating an example of an ultrasonic image generated through a BF process.

FIG. 4 is a diagram illustrating an example of an ultrasonic image generated through an adaptive BF process.

FIG. 5 is a schematic diagram illustrating another example of a scanning range.

FIG. 6 is a diagram illustrating another example of an ultrasonic image generated through the BF process.

FIG. 7 is a diagram illustrating another example of another ultrasonic image generated through the adaptive BF process.

FIG. 8 is a block diagram illustrating a functional configuration example of an ultrasonic measurement apparatus in a first embodiment.

FIG. 9 is a flowchart illustrating a flow of an ultrasonic image generation process in the first embodiment.

FIG. 10 is a diagram illustrating an example of an edge detection result.

FIG. 11 is a diagram illustrating an example of a beam-direction orthogonal line excluding process result.

FIG. 12 is a diagram illustrating a labeling process result.

FIG. 13 is a diagram illustrating a result of extracting a beam-direction non-orthogonal line.

FIG. 14 is a schematic diagram illustrating still another example of a scanning range.

FIG. 15 is a diagram illustrating still another example of an ultrasonic image generated through the BF process.

FIG. 16 is a diagram illustrating still another example of another ultrasonic image generated through the adaptive BF process.

FIG. 17 is a block diagram illustrating a functional configuration example of an ultrasonic measurement apparatus in a second embodiment.

FIG. 18 is a diagram illustrating a data configuration example of a non-orthogonal line candidate list.

FIG. 19 is a flowchart illustrating a flow of an ultrasonic image generation process in the second embodiment.

### DESCRIPTION OF EXEMPLARY EMBODIMENTS

Hereinafter, a preferred embodiment of the invention will be described with reference to the drawings. The invention is not limited to embodiments described below, and forms to which the invention is applicable are not limited to the following embodiments. The same constituent elements are given the same reference numerals throughout the drawings.

### First Embodiment

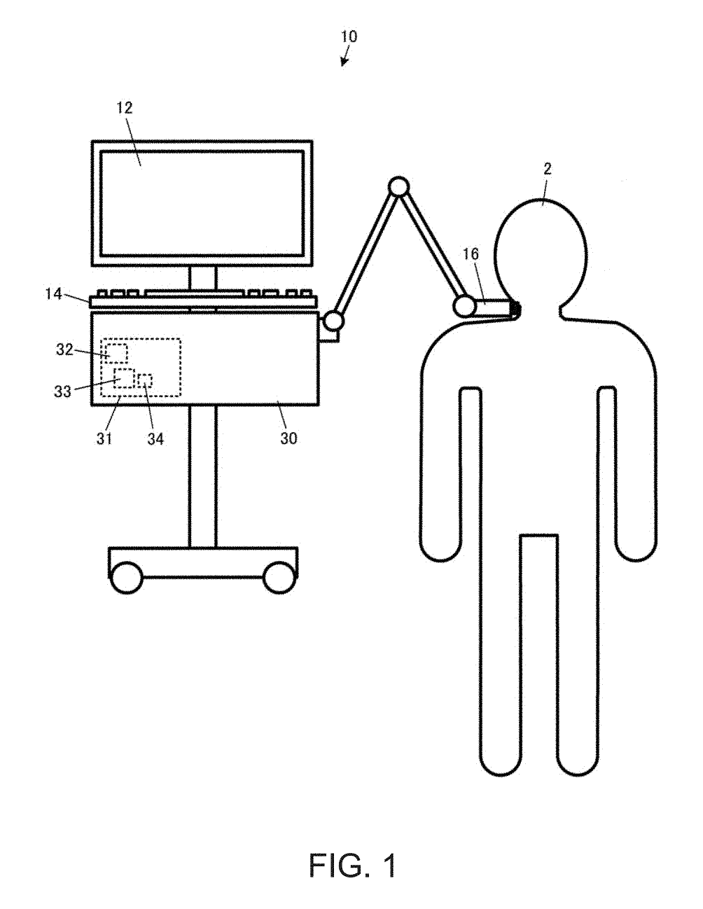

FIG. 1 is a diagram illustrating a system configuration example of an ultrasonic measurement apparatus in a first embodiment. The ultrasonic measurement apparatus 10 is used to acquire biological information of a subject 2 through ultrasonic measurement, and includes a touch panel 12 which displays a measurement result or operation information as an image and also receives an operation input, a keyboard 14 which receives an operation input, an ultrasonic probe 16 capable of performing linear scanning, and a processing device 30.

A control board 31 is mounted on the processing device 30, and is connected to the respective apparatus units such as the touch panel 12, the keyboard 14, and the ultrasonic probe 16 so as to transmit and receive signals therewith. The control board 31 is mounted with a central processing unit (CPU) 32, various integrated circuits such as an application specific integrated circuit (ASIC) or a field-programmable gate array (FPGA), a storage medium 33 formed of an IC memory or a hard disk, and a communication IC 34 which performs data communication with external devices. In the ultrasonic measurement apparatus 10, the CPU 32 or the like of the processing device 30 executes a program stored in the storage medium 33 so as to perform processes required to acquire biological information, including ultrasonic measurement.

Specifically, the ultrasonic measurement apparatus 10 transmits an ultrasonic beam to the subject 2 from the ultrasonic probe 16, and receives a reflected wave thereof so as to perform ultrasonic measurement, under the control of the processing device 30. The ultrasonic measurement apparatus 10 performs amplification and signal processing on a received signal of the reflected wave so as to generate reflected wave data such as position information or temporal changes of a biological structure of the subject 2. The ultrasonic measurement is repeatedly performed in a predetermined cycle. The measurement unit is referred to as a “frame”.

The reflected wave data includes images in respective modes such as so-called A mode, B mode, M mode, and color Doppler mode. The A mode is a mode in which a first axis expresses a sampling point sequence of received signals along transmission and reception directions (directions of scanning lines) of ultrasonic beams, a second axis expresses the received signal intensity (reflected wave intensity) of a reflected wave at each sampling point, and the amplitude of a reflected wave (A mode image) is displayed. The B mode is a mode in which a two-dimensional ultrasonic image (B mode image) of a biological structure which is visualized by converting, with a luminance value, a reflected wave amplitude (A mode image) obtained by applying an ultrasonic beam within a predetermined scanning range.

### Principle

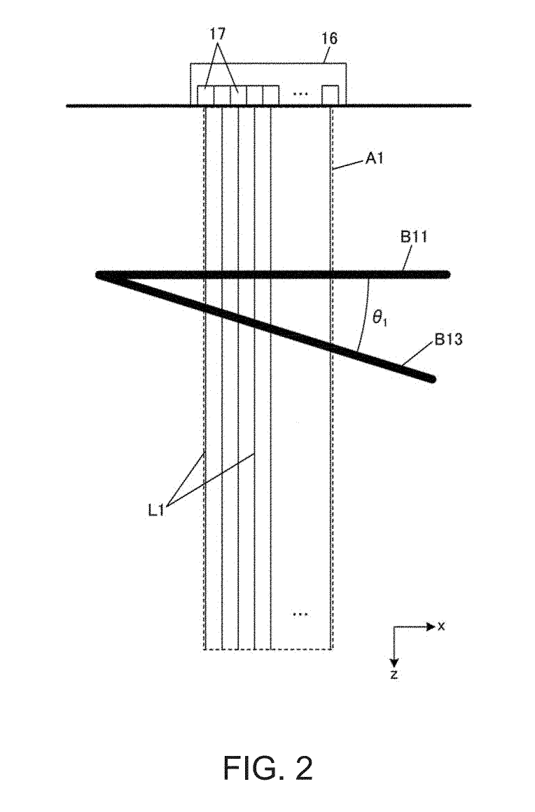

FIG. 2 is a diagram for explaining ultrasonic measurement in a first embodiment, and schematically illustrates a state in which the ultrasonic probe 16 is brought into contact with a surface of a target object, and ultrasonic measurement is performed. The ultrasonic probe 16 is provided with ultrasonic elements (ultrasonic vibrators) disposed in a line at the same interval therein, and a group of a plurality of ultrasonic elements adjacent to each other forms a single channel 17 so as to transmit and receive ultrasonic waves. In the first embodiment, if incident positions of ultrasonic beams are deviated in an arrangement direction (scanning direction) of the channel 17, ultrasonic beams are transmitted and received along a plurality of scanning lines L1 which are parallel to each other, and thus ultrasonic measurement is performed according to a so-called linear scanning method in which scanning is performed in a rectangular scanning range A1.

In actual ultrasonic measurement performed by the ultrasonic measurement apparatus 10, the ultrasonic probe 16 is brought into contact with a living body surface (the neck in FIG. 1) of the subject 2, and reflected wave data of a target region corresponding to the scanning range is generated. A horizontal direction of the target region (scanning range) is defined as an x direction, and a vertical direction of the target region orthogonal thereto is defined as a z direction. The x direction corresponds to a scanning direction, and the z direction corresponds to a depth direction from the living body surface. A part with which the ultrasonic probe 16 is brought into contact is not limited the neck, and may be a part of the subject 2 depending on the measurement (diagnosis) purpose, such as the wrist, the arm, and the abdomen.

When reflected wave data is generated from a received signal (hereinafter, referred to as a “channel signal”) which is received by each channel 17 as a result of the ultrasonic measurement, the processing device 30 performs a process of performing phasing addition on channel signals from the respective channels 17 for each sampling point. In a case where ultrasonic waves are transmitted and received in the element unit, phasing addition is performed on received signals of reflected waves from the respective channels 17. Through this process, a channel signal of each channel 17 is regarded as two-dimensional data indicating a reflected wave intensity at each sampling point, a necessary process such as a detection process or a logarithmic conversion process is performed on the two-dimensional data such that a luminance value is obtained for each sampling point, and thus an ultrasonic image of the target region can be generated. There may be a configuration in which phasing addition is performed on received signals which are received by respective ultrasonic elements instead of the channel unit, and thus an ultrasonic image is generated.

Specifically, the phasing addition process is a signal combination process in which all of the scanning lines L1 are sequentially processing targets, and channel signals from the respective channels 17 are combined with each other for each sampling point on a processing target scanning line (processing target line) L1, a delay process of delaying a channel signal of each channel 17 is performed, and then a reception beam forming process (reception BF process) of performing weighted addition of respective channel signals after the delay process is performed. Consequently, only signals with the same phase from a desired direction can be amplified, and thus a desired wave from a direction of the processing target line L1 can be extracted.

Here, in the related art, as the reception BF process, there is an adaptive beam forming process (adaptive BF process) of dynamically changing a weight (signal combination weight) used for addition of channel signals depending on an incoming wave. In the adaptive BF process, the following process is performed for each sampling point. That is, first, a correlation matrix is calculated on the basis of a channel signal of each channel 17 after a delay process. Next, an adaptive weight Wmv multiplied by each channel signal is calculated from the calculated correlation matrix by using a steering vector defined on the basis of the direction of the scanning line L1. Calculation of the adaptive weight Wmv will be described later with reference to Equations (1) to (8). The channel signals of the respective channels 17 after the delay process are subjected to weighted addition by using the calculated adaptive weight Wmv as a signal combination weight W. A specific example of the adaptive BF process includes a minimum variance (MV) method or an amplitude and phase estimation (APES) method, which may be employed as appropriate. According to the adaptive BF process, channel signals can be subjected to weighted addition by adding a constraint to a direction such that the sensitivity for only a desired wave from a direction of the processing target line L1 is provided, and the sensitivity for an unnecessary wave from other directions is not provided, and thus it is possible to realize a high azimuth resolution.

As another reception BF process, there is a non-adaptive reception BF process (hereinafter, this non-adaptive reception BF process will be simply referred to as a “BF process”) in which a predetermined weight (hereinafter, referred to as a “fixed weight”) Wf defined in correlation with each channel 17 in advance is used as the signal combination weight W. The BF process is advantageous in that a process can be performed in a short period of time since calculation of a weight is not necessary, but the sensitivity for an unnecessary wave is also provided, and thus an azimuth resolution is lower than that in the adaptive BF process.

A reflected wave intensity of an ultrasonic wave obtained as a received signal increases at a position where media through which the ultrasonic wave propagates change, and a position where the reflected wave intensity is high in an ultrasonic image is displayed in high luminance. Therefore, it is possible to recognize an interface of a living tissue or the like present under the skin by observing an ultrasonic image of a target region. However, the visibility of a depicted interface differs in cases where the reception BF process is performed as the adaptive BF process and the BF process, and thus there is the following problem in visibility.

In other words, in a case where a direction of the scanning line L1 which is a beam direction in the reception BF process is orthogonal or substantially orthogonal to an interface, the interface can be clearly depicted by applying the adaptive BF process. However, in a case where an interface which is not orthogonal to the beam direction is present in a target region, as an angle formed between the interface and the x direction increases, the visibility thereof deteriorates in a generated ultrasonic image. This phenomenon also occurs in both of the cases where the BF process and the adaptive BF process are applied, but more remarkably occurs in a case where the adaptive BF process is applied.

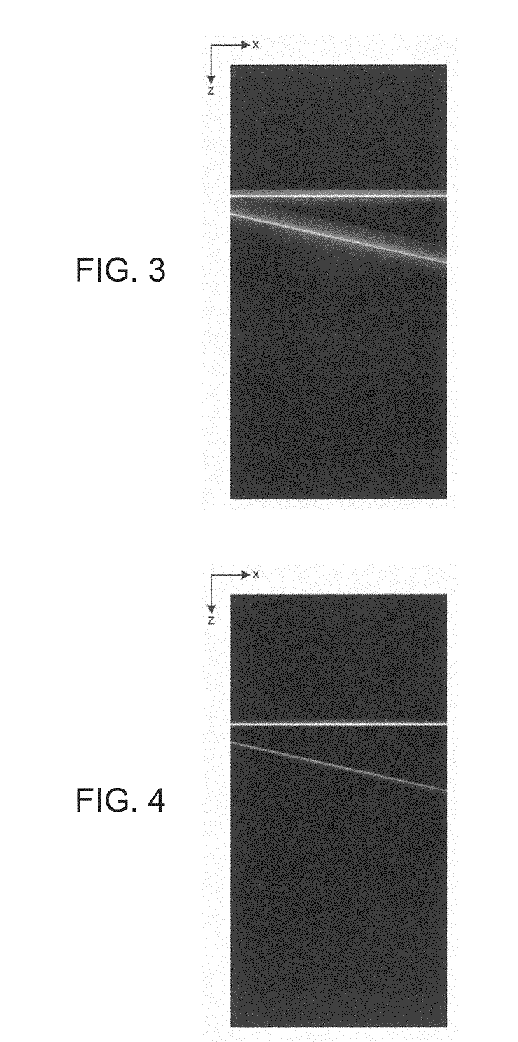

For comparative examination, as illustrated in FIG. 2, two wires B11 and B13 were disposed in the scanning range A1, and an ultrasonic image of the scanning range A1 in a case where ultrasonic measurement was performed was generated through simulation. One wire B11 is disposed along the x direction orthogonal to the beam direction, and the other wire B13 is disposed to be inclined with respect to the x direction at an angle of θ₁ formed with the x direction. FIG. 3 is a diagram illustrating an ultrasonic image generated in a case where the BF process is performed as the reception BF process, and FIG. 4 is a diagram illustrating an ultrasonic image generated in a case where the adaptive BF process is performed as the reception BF process. As illustrated in FIGS. 3 and 4, the adaptive BF process is applied to the one wire B11 orthogonal to the beam direction, and thus one wire B11 is clearly depicted. However, focusing on the other wire B13 which is not orthogonal to the beam direction, in a case where the adaptive BF process is applied thereto, the luminance of the wire B13 is lower and is thus hardly viewed than in a case where the BF process is applied, and thus the visibility thereof deteriorates.

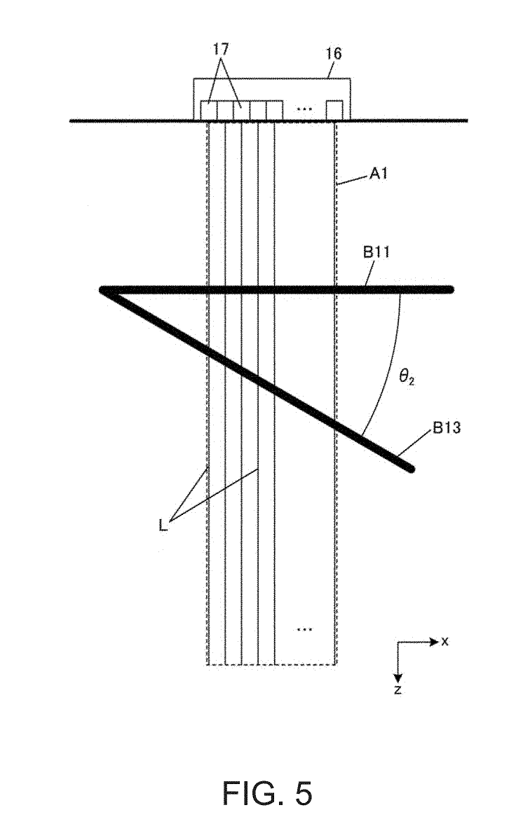

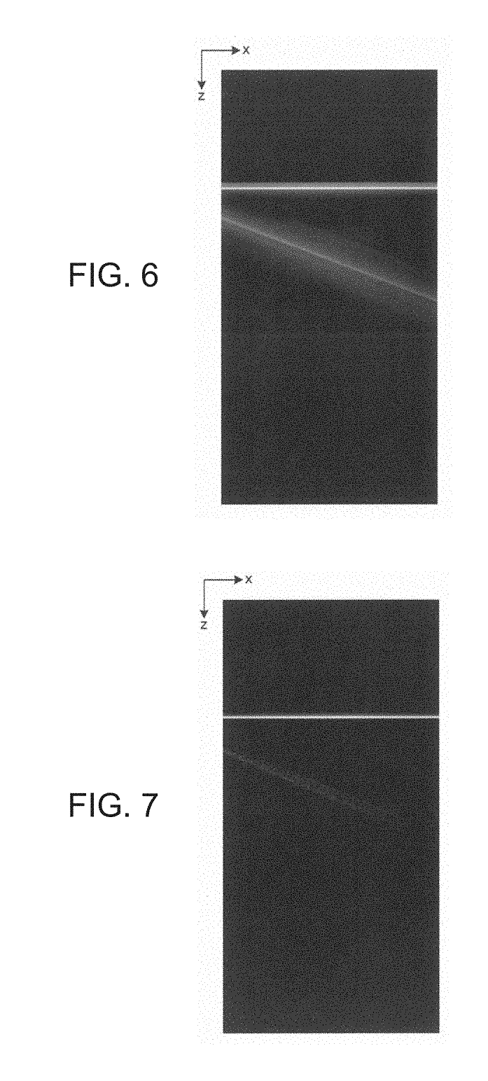

Next, as illustrated in FIG. 5, ultrasonic measurement was performed in a state in which the other wire B13 is further inclined with respect to the x direction at an angle of θ₂ (θ₂>θ₁) formed between the wire B13 and the x direction, and an ultrasonic image of the scanning range A1 was generated. FIG. 6 illustrates an ultrasonic image generated in a case where the reception BF process is performed as the BF process in this example, and FIG. 7 is a diagram illustrating an ultrasonic image generated in a case where the reception BF process is performed as the adaptive BF process. In a case of this example, as illustrated in FIG. 7, the wire B13 is scarcely depicted in the image to which the adaptive BF process is applied, and is hardly visually recognized. In contrast, as illustrated in FIG. 6, the entire region can be visually recognized in a case where the BF process is applied.

As mentioned above, in a case where the reception BF process is performed as the adaptive BF process, there is a problem in that the visibility of an extracted interface which is not orthogonal to the beam direction considerably deteriorates. Therefore, in the first embodiment, weight selection is performed such that the fixed weight Wf is used as the signal combination weight W with respect to a sampling point of an interface which is not orthogonal to the beam direction. With respect to other sampling points, the adaptive weight Wmv is calculated, and weight selection is performed such that the adaptive weight Wmv is used as the signal combination weight W.

Regarding a process for this, first, edge detection is performed with respect to a target region. An edge portion (hereinafter, referred to as a “beam-direction non-orthogonal line” as appropriate) which is not orthogonal to the beam direction is extracted among detected edge portions. Regarding procedures, for example, among the detected edge portions, an edge portion (that is, an edge portion orthogonal to the beam direction; hereinafter, referred to as a “beam-direction orthogonal line” as appropriate) of which an extension direction is parallel to a scanning direction in linear scanning is set as an extraction exclusion target. An edge portion satisfying a predetermined condition is extracted from the edge portions which are not the exclusion targets as a beam-direction non-orthogonal line, the predetermined condition being that “the edge portion is not orthogonal to the beam direction, and has a length of a predetermined length D_{E} set in advance or more”.

Thereafter, each sampling point on the beam-direction non-orthogonal line is set as a BF process position, and, in a case where a processing target point is the BF process position, the fixed weight Wf is used as the signal combination weight W. In a case where a processing target point is not the BF process position, the adaptive weight Wmv is calculated to be used as the signal combination weight W.

### Functional Configuration

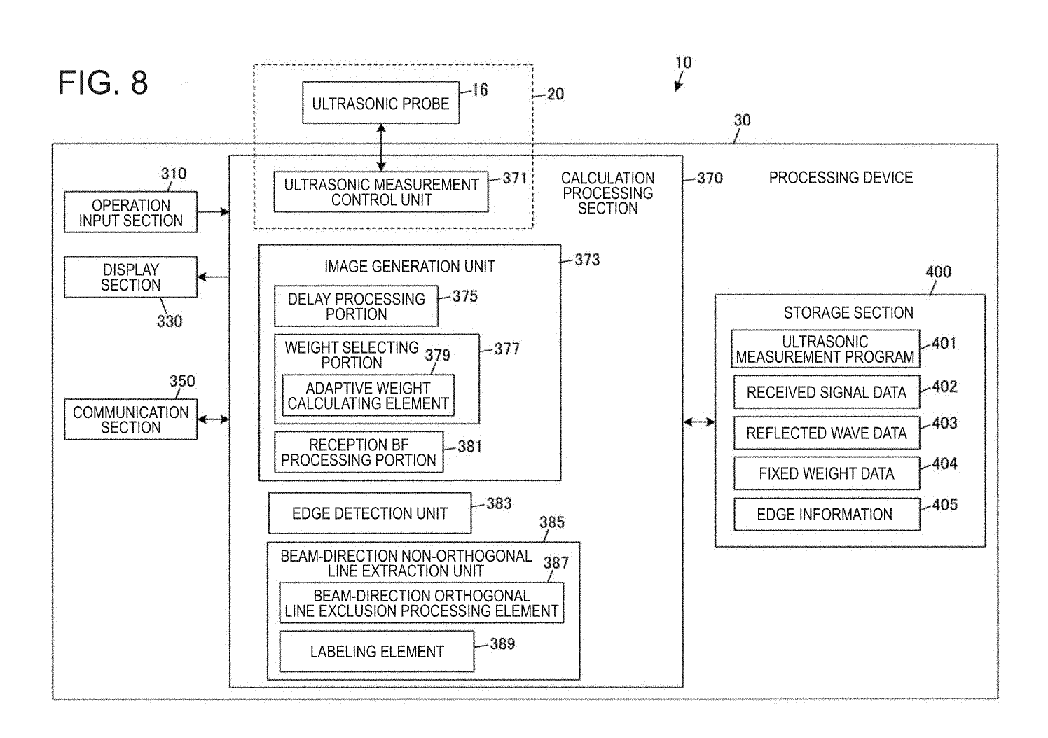

FIG. 8 is a block diagram illustrating a functional configuration example of the ultrasonic measurement apparatus in the first embodiment. The ultrasonic measurement apparatus 10 includes the processing device 30 and the ultrasonic probe 16. The processing device 30 includes an operation input section 310, a display section 330, a communication section 350, a calculation processing section 370, and a storage section 400.

The ultrasonic probe 16 includes the channels 17 which are formed of a plurality of ultrasonic element groups and are arranged, and transmits an ultrasonic wave on the basis of a pulse voltage from the processing device 30 (more specifically, an ultrasonic measurement control unit 371 of the calculation processing section 370). A reflected wave of the transmitted ultrasonic wave is received, and a channel signal which is a received signal of each channel 17 is output to the ultrasonic measurement control unit 371.

The operation input section 310 receives various operation inputs performed by authority, and outputs operation input signals corresponding to the operation inputs to the calculation processing section 370. The operation input section 310 may be implemented by a button switch, a lever switch, a dial switch, a track pad, a mouse, and the like. In FIG. 1, the touch panel 12 or the keyboard 14 corresponds to the operation input section 310.

The display section 330 is implemented by a display device such as a liquid crystal display (LCD), and performs various displays based on display signals from the calculation processing section 370. In FIG. 1, the touch panel 12 corresponds to the display section 330.

The communication section 350 is a communication device which transmits and receives data to and from external devices under the control of the calculation processing section 370. A communication method of the communication section 350 may employ various methods such as a format of wired connection using a cable based on a predetermined communication standard, a format of connection using an intermediate device which is also used as a charger called a cradle, and a format of wireless connection using wireless communication. In FIG. 1, the communication IC 34 corresponds to the communication section 350.

The calculation processing section 370 is implemented by, for example, a microprocessor such as a CPU or a graphics processing unit (GPU), and electronic components such as an ASIC, an FPGA, and an IC memory. The calculation processing section 370 controls input and output of data between the respective functional units, and performs various calculation processes on the basis of a predetermined program or data, an operation input signal from the operation input section 310, a channel signal of each channel 17 from the ultrasonic probe 16, and the like, so as to calculate biological information of the subject 2. In FIG. 1, the CPU 32 corresponds to the calculation processing section 370. The calculation processing section 370 may be formed of hardware such as a dedicated module circuit.

The calculation processing section 370 includes the ultrasonic measurement control unit 371, an image generation unit 373, an edge detection unit 383, and a beam-direction non-orthogonal line extraction unit 385.

The ultrasonic measurement control unit 371 forms an ultrasonic measurement section 20 along with the ultrasonic probe 16, and the ultrasonic measurement section 20 performs ultrasonic measurement. The ultrasonic measurement control unit 371 may be realized by using a well-known technique. In other words, the ultrasonic measurement control unit 371 controls a timing at which the ultrasonic probe 16 transmits an ultrasonic pulse, generates a pulse voltage at the transmission timing, and outputs the pulse voltage to the ultrasonic probe 16. At this time, a transmission delay process is performed, and thus a timing at which the pulse voltage is output to each ultrasonic element is adjusted. An amplification process or a filtering process is performed on a channel signal from each channel 17, and the channel signal (measurement result) of each channel 17 after the process is output to the image generation unit 373.

The image generation unit 373 generates an ultrasonic image on the basis of channel signals of the respective channels 17 from the ultrasonic measurement control unit 371. The image generation unit 373 includes a delay processing portion 375, a weight selecting portion 377, and a reception BF processing portion 381.

The delay processing portion 375 performs a delay process of delaying a channel signal of each channel 17 by a delay time defined in advance in correlation with each channel 17.

The weight selecting portion 377 performs weight selection of setting the fixed weight Wf or the adaptive weight Wmv as the signal combination weight W for each sampling point. In other words, in a case where a processing target point is a BF process position, the fixed weight Wf is selected as the signal combination weight W, and, in a case where a processing target point is not a BF process position, the adaptive weight Wmv is selected as the signal combination weight W. The weight selecting portion 377 includes an adaptive weight calculating element 379 calculating the adaptive weight Wmv.

Here, a description will be made of calculation of the adaptive weight Wmv performed by the adaptive weight calculating element 379. In a case where a signal combination weight corresponding to each channel 17 is indicated by Wₘ, an output z[n] from the reception BF processing portion 381 is a result of adding together results obtained by multiplying channel signals xₘ[n−Dₘ[n]] of the respective channels 17 after a delay process, output from the delay processing portion 375 by the corresponding signal combination weight Wₘ of the channels 17, and is expressed by the following Equation (1). In Equation (1), m indicates the number of channels. In addition, n indicates a total number of times of sampling, and xₘ[n] indicates a channel signal at a sampling point n of an m-th channel 17. Here, Dₘ indicates a delay time which is applied to the m-th channel 17 in a delay process in the previous stage. In other words, in a case where the signal combination weight Wₘ used here is the adaptive weight Wmv (Wmvₘ) calculated for the respective channels 17, the adaptive BF process is performed as the reception BF process, and, in a case of the fixed weight Wf (Wfₘ) defined in advance in correlation with each channel 17, the BF process is performed as the reception BF process.

$$z [ n ] = \frac{1}{M} {\sum }_{m = 0}^{M - 1} {W}_{m} [ n ] {x}_{m} [ n - {D}_{m} [ n ] ] \qquad ( 1 )$$

Meanwhile, in a case where the adaptive weight Wmv is calculated to be used as the signal combination weight W, if Wₘ is replaced with the adaptive weight Wmvₘ of each channel 17, and the output z[n] in Equation (1) is expressed in a vector, this leads to the following Equations (2) and (3). H indicates a complex conjugate transposition, and * indicates a complex conjugate.

$$\begin{matrix}z [ n ] = \frac{1}{M} {Wmv [ n ]}^{H} X [ n ] & ( 2 ) \\ Wmv [ n ] = [ \begin{matrix}{Wmv}_{0}^{*} [ n ] \\ {Wmv}_{1}^{*} [ n ] \\ ⋮ \\ {Wmv}_{M - 1}^{*} [ n ]\end{matrix} ] & ( 3 )\end{matrix}$$

A correlation matrix R[n] is given by the following Equations (4) and (5).

R[n]=E[X[n]X[n]^{T}] (4)

z[|x[n]|²]=Wmv[n]^{H}R[n]Wmv[n] (5)

The adaptive weight Wmvₘ of each channel 17 is a value minimizing the variance of z[n] in above Equations (4) and (5), and is obtained by solving a conditional miniaturization problem expressed in the following Equations (6) and (7). If a Lagrange multiplier method is used, the adaptive weight Wmvₘ of each channel 17 is expressed by the following Equation (8).

$$\begin{matrix}{min}_{w [ n ]} {Wmv [ n ]}^{H} R [ n ] Wmv [ n ] & ( 6 ) \\ subject to {Wmv [ n ]}^{H} a = 1 & ( 7 ) \\ Wmv [ n ] = \frac{{R [ n ]}^{- 1} a}{{a}^{H} {R [ n ]}^{- 1} a} & ( 8 )\end{matrix}$$

Here, a indicates a steering vector, and H indicates a restraint response vector. A channel signal is already phases in the delay process in the previous stage, and thus a direction of the desired wave is 0 degrees. Therefore, all elements of the vector a may be 1.

FIG. 8 is referred to again. The reception BF processing portion 381 performs the reception BF process of performing weighted addition on the channel signals of the respective channels 17 after the delay process according to Equation (1) by using the signal combination weight W determined by the weight selecting portion 377.

The edge detection unit 383 performs a well-known edge detection process so as to detect edge portions from a target region.

The beam-direction non-orthogonal line extraction unit 385 extracts edge portions (beam-direction non-orthogonal lines) which are not orthogonal to the beam direction and have a length of the predetermined length D_{E} or more from the edge portions detected by the edge detection unit 383. The beam-direction non-orthogonal line extraction unit 385 includes a beam-direction orthogonal line exclusion processing element 387 and a labeling element 389. The beam-direction orthogonal line exclusion processing element 387 performs a process of excluding an edge portion (beam-direction orthogonal line) orthogonal to the beam direction from an extraction target of the beam-direction non-orthogonal line among the edge portions detected by the edge detection unit 383. The labeling element 389 performs a well-known labeling process, and adds a unique label to each edge portion which is not excluded by the beam-direction orthogonal line exclusion processing element 387.

The storage section 400 is implemented by storage medium such as an IC memory, a hard disk, or an optical disc. The storage section 400 stores a program for operating the ultrasonic measurement apparatus 10 so as to realize various functions of the ultrasonic measurement apparatus 10, and stores data used during execution of the program in advance or temporarily stores data whenever a process is performed. In FIG. 1, the storage medium 33 mounted on the control board 31 corresponds to the storage section 400. Connection between the calculation processing section 370 and the storage section 400 is not limited to connection using an internal bus circuit in the apparatus, and may be realized a communication line such as a local area network (LAN) or the Internet. In this case, the storage section 400 may be implemented by an external storage device separate from the ultrasonic measurement apparatus 10.

The storage section 400 stores an ultrasonic measurement program 401, received signal data 402, reflected wave data 403, fixed weight data 404, and edge information 405.

The calculation processing section 370 reads and executes the ultrasonic measurement program 401 so as to realize functions of the ultrasonic measurement control unit 371, the image generation unit 373, the edge detection unit 383, the beam-direction non-orthogonal line extraction unit 385, and the like. In a case where such functional units are realized by hardware such as an electronic circuit, some of programs for realizing the functions may be omitted.

A channel signal from each channel 17, obtained as a result of ultrasonic measurement, is stored in the received signal data 402 for each frame.

Reflected wave data obtained through ultrasonic measurement which is repeatedly performed for each frame is stored in the reflected wave data 403. The reflected wave data 403 includes data of a B mode image for each frame as an ultrasonic image.

The fixed weight Wf is stored in the fixed weight data 404. The fixed weight Wf may be defined by using, for example, a window function such as a square window (rectangular window), a Hanning window, or a Hamming window. For example, in a case of using the square window, as shown in the following Equation (9), the fixed weight Wf is set in the fixed weight data 404 such that the fixed weight Wfₘ of the respective channels 17 are all “1”.

Wfₘ={1,1, . . . ,1} (9)

An edge detection result in the edge detection unit 383 is stored in the edge information 405. The edge information 405 is rewritten by the beam-direction non-orthogonal line extraction unit 385 in the process of extracting a beam-direction non-orthogonal line as will be described later.

### Flow of Process

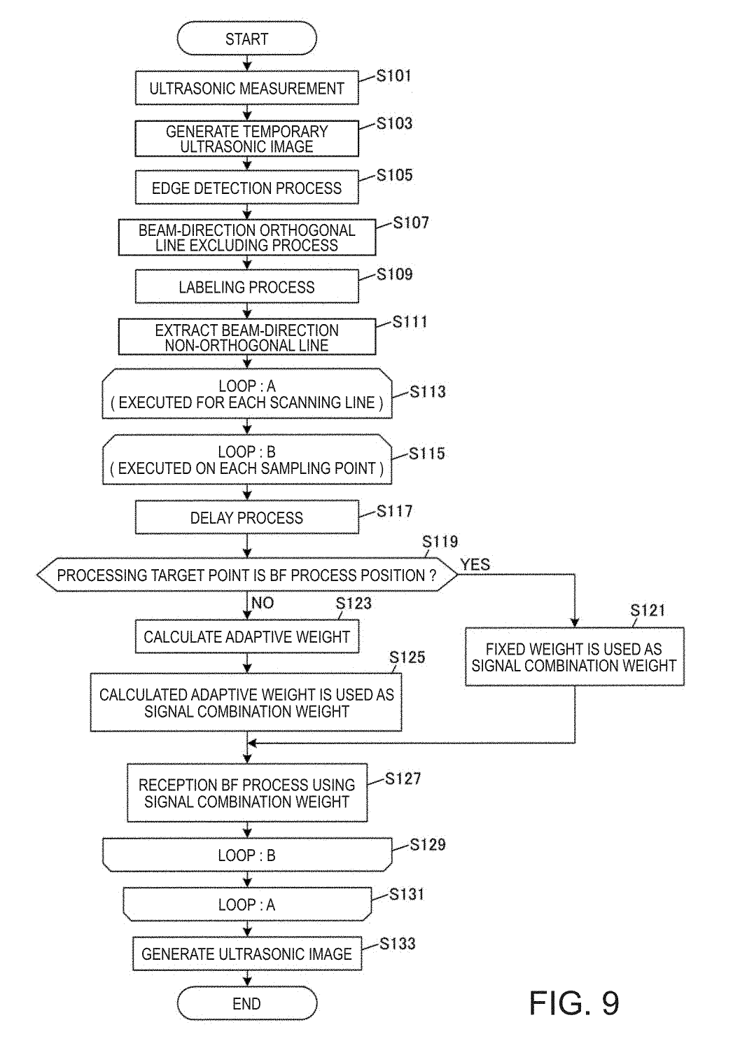

FIG. 9 is a flowchart illustrating a flow of an ultrasonic image generation process in the first embodiment. The process described here is started, for example, if the ultrasonic probe 16 is brought into contact with a living body surface of the subject 2 by a user, and a predetermined measurement starting operation is performed. This process may be realized by the calculation processing section 370 reading the ultrasonic measurement program 401 from the storage section 400 and executing the ultrasonic measurement program 401 so as to operate the respective sections of the ultrasonic measurement apparatus 10.

First, the ultrasonic measurement section 20 performs ultrasonic measurement (step S101). A measurement result is stored in the received signal data 402 through this process.

Next, the image generation unit 373 generates a temporary ultrasonic image (step S103). Herein, an ultrasonic image is generated according to a method of the related art. For example, first, the delay processing portion 375 performs a process of delaying a channel signal from each channel 17 obtained through the ultrasonic measurement in step S101. The reception BF processing portion 381 performs the BF process as the reception BF process. In other words, weighted addition is performed on channel signals of the respective channels 17 for each sampling point after the delay process according to the above Equation (1) by using the fixed weight Wf as the signal combination weight W. Thereafter, a necessary process is performed on the output z[n] from the reception BF processing portion 381 such that a luminance value of each sampling point is obtained.

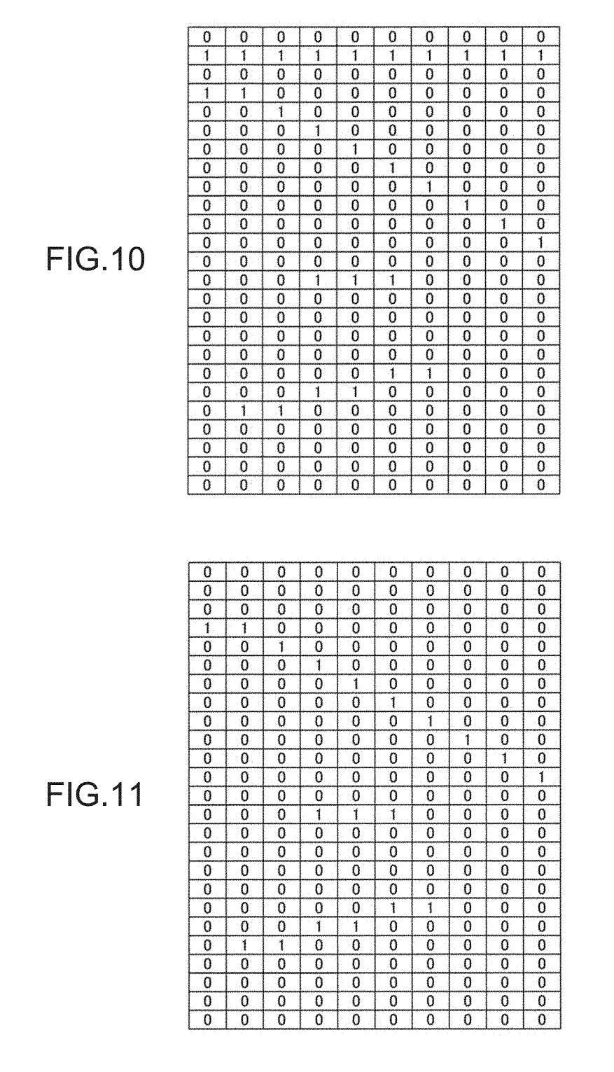

Next, the edge detection unit 383 performs an edge detection process on the temporary ultrasonic image so as to detect edge portions present in a target region (step S105). Specifically, a differential value is calculated for each sampling point by applying a well-known differential filter for detecting horizontal edges including an edge portion in a horizontal direction (x direction) and an edge portion in an inclined direction. The edge information 405 is generated such that a value for a sampling point at which an obtained differential value is equal to or greater than a predetermined threshold value set in advance is “1”, and a value for a sampling point at which the obtained differential value is smaller than the threshold value is “0”. FIG. 10 illustrates an example of the edge information 405.

Herein, the edge detection process is performed on the temporary ultrasonic image generated in step S103 such that edge portions present in the target region are detected. In contrast, an edge detection process may be performed on two-dimensional data (the output z[n] from the reception BF processing portion 381) after a reception BF process such that edge portions present in the target region are detected.

Next, as illustrated in FIG. 9, the beam-direction orthogonal line exclusion processing element 387 performs a beam-direction orthogonal line excluding process such that an edge portion in the x direction is erased from the edge information 405 generated through the edge detection process (step S107). Regarding a procedure, first, a position (x,y) of each sampling point of the edge information 405 illustrated in FIG. 10 is sequentially referred to by one row such that a range in which “1” continues in each row is specified, and the number of continuing is (hereinafter, simply referred to as a “continuation number”) is counted. A range in which the counted continuation number is equal to or larger than a predetermined number set in advance is determined as being an edge portion, and a value of a sampling point in the range is rewritten to “1” from “0” such that the edge information 405 is updated. Consequently, the edge portion in the x direction is erased from the edge information 405. FIG. 11 illustrates the edge information 405 after the beam-direction orthogonal line excluding process. In the example illustrated in FIG. 11, the edge portion in the second row from the top of the edge information 405 in FIG. 10 is erased.

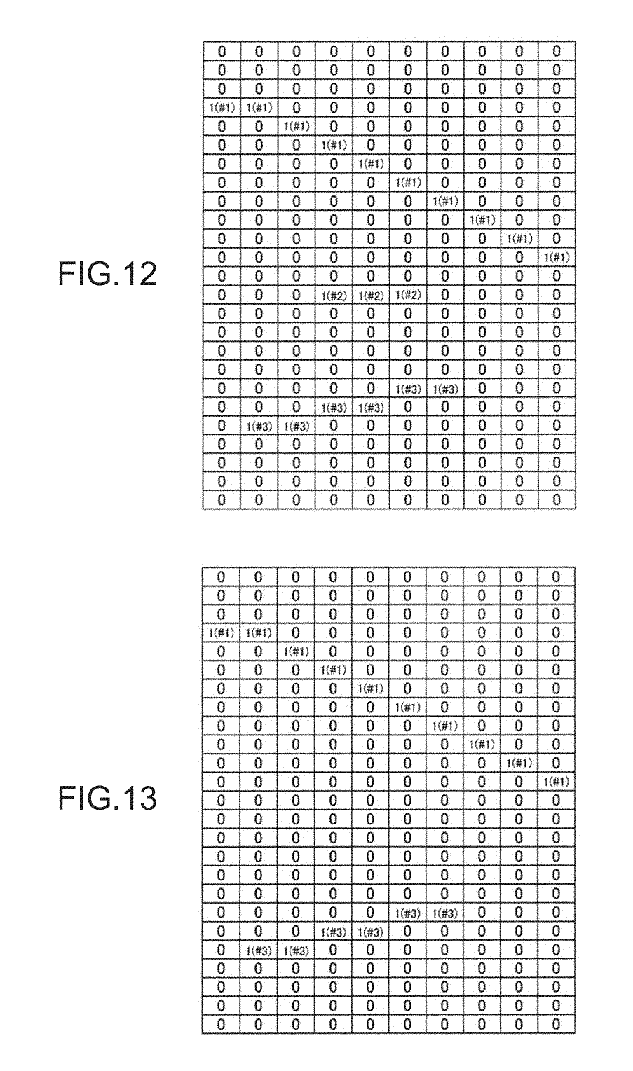

Next, as illustrated in FIG. 9, the labeling element 389 performs a labeling process on the edge information 405 after the beam-direction orthogonal line excluding process (step S109). Regarding a procedure, first, a sampling point of which a value is “1” in the edge information 405 in FIG. 11 is divided as a sampling point group in which “1s” are adjacent to each other. Herein, in a case where eight sampling points of which a value is “1” are present near a focused sampling point, the sampling points are connected to each other such that the sampling point group is obtained. A unique label for each of divided sampling points is added thereto. FIG. 12 illustrates the edge information 405 after the labeling process. In the example illustrated in FIG. 12, labels of label numbers “1” to “3” are respectively added to sampling point groups forming three edge portions. A sampling method is not particularly limited, and a divided sampling point group may be obtained by referring to four peripheral values.

Thereafter, as illustrated in FIG. 9, the beam-direction non-orthogonal line extraction unit 385 erases an edge portion of below the predetermined length D_{E} from the edge information 405 after the labeling process so as to extract a beam-direction non-orthogonal line (step S111). Regarding a procedure, first, the number of sampling points of edge portions is counted for the edge portions added with the same label. With respect to an edge portion in which a count value is less than a predetermined number indicating the predetermined length D_{E}, a value of each sampling point is rewritten to “0” such that the edge information 405 is updated. Consequently, the edge portion of below the predetermined length D_{E} is erased from the edge information 405. FIG. 13 illustrates a beam-direction non-orthogonal line extraction result (edge information 405). In the example illustrated in FIG. 13, the edge portion with the label number “2”, formed of three sampling points is erased from the edge information 405 in FIG. 12, and, finally, the edge portions with the label numbers and “3” are regarded as beam-direction non-orthogonal lines.

The above-described processes related to extraction of a beam-direction non-orthogonal line may be performed by thinning out the edge information 405 as appropriate for simplification thereof.

Referring to FIG. 9 again, in a case where the beam-direction non-orthogonal lines are extracted in the above-described way, the image generation unit 373 repeatedly performs a loop A for each scanning line L1 by referring to the received signal data 402 (step S113 to step S131). In the loop A, the processing target line L1 is sampled at a predetermined time interval by using the measurement result of the ultrasonic measurement in step S101, and a process in a loop B is sequentially performed with each sampling point as a processing target point (step S115 to step S129).

In the loop B, first, the delay processing portion 375 performs a delay process of delaying a channel signal of each channel 17 by a delay time (step S117).

Next, the weight selecting portion 377 refers to the edge information 405, determines a processing target point as being a BF process position if a value of the processing target point is “1” (step S119: YES), and proceeds to step S121. In other words, in step S121, the weight selecting portion 377 reads the fixed weight Wf from the fixed weight data 404 so as to use the fixed weight Wf as the signal combination weight W. On the other hand, in a case where a value of a processing target point is “0” in the edge information 405 (step S119: NO), the adaptive weight calculating element 379 of the weight selecting portion 377 calculates the adaptive weight Wmv according to the above-described method on the basis of a channel signal of each channel 17 after the delay process (step S123). The weight selecting portion 377 uses the calculated adaptive weight Wmv as the signal combination weight W (step S125).

Thereafter, the reception BF processing portion 381 performs the reception BF process by using the fixed weight Wf or the adaptive weight Wmv as the signal combination weight W in step S121 or step S125, and performs weighted addition on channel signals of the respective channels 17 after the delay process according to the above Equation (1) (step S127).

In a case where the process in this loop B is repeatedly performed, and sampling of the processing target line L1 is completed, the process in the loop A on the processing target line L1 is completed. In a case where the process in the loop A has been performed with all of the scanning lines L1 as processing targets, a necessary process is performed on the output z[n] from each sampling point such that an ultrasonic image is generated (step S133), and the present process is finished. The generated ultrasonic image is controlled to be displayed on the display section 330 as appropriate.

As described above, according to the first embodiment, edge detection can be performed with respect to a target region, and an edge portion which is not orthogonal to a beam direction and has a length of the predetermined length D_{E} or more can be extracted from detected edge portions. In a case where a processing target point is an extracted edge portion, the reception BF process is performed by using the fixed weight Wf as the signal combination weight W, and, in a case where a processing target point is not an extracted edge portion, the reception BF process is performed by using the adaptive weight Wmv as the signal combination weight W, such that an ultrasonic image can be generated. Therefore, it is possible to maintain a high azimuth resolution when an ultrasonic image is generated by scanning a target region according to a linear scanning method, and also to improve the visibility of an interface in a case where there is the interface which is not orthogonal to a beam direction in the target region.

### Second Embodiment

Next, a second embodiment will be described. In the following description, the same constituent elements as in the first embodiment are given the same reference numerals.

### Principle

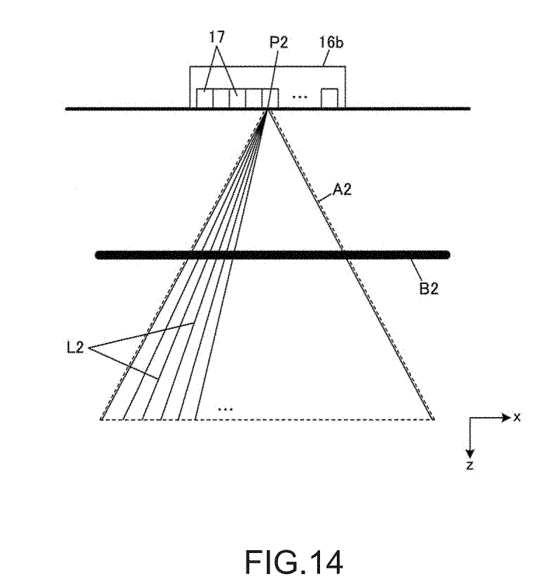

FIG. 14 is a diagram for explaining ultrasonic measurement in the second embodiment. In the second embodiment, an ultrasonic probe 16b performs ultrasonic measurement according to a sector scanning method. In other words, in the sector scanning method, ultrasonic beams are radially transmitted and received along a plurality of scanning lines L2 with a predetermined target object surface position as a starting point P2 while changing an incidence angle by a predetermined angle, and sector scanning is performed in a predetermined angle range (scanning range) A2. In actual ultrasonic measurement performed by an ultrasonic measurement apparatus 10b of the second embodiment, the ultrasonic probe 16b is brought into contact with a living body surface (for example, the neck) of the subject 2, and reflected wave data of a target region corresponding to the scanning range is generated.

In the sector scanning method, as mentioned above, since ultrasonic beams are radially transmitted and received in the directions of the respective scanning lines L2, in a case where an interface is present in a target region, a relationship between the interface and a beam direction differs for each beam direction. In other words, even for the same interface, a beam direction which is a direction of the scanning line L2 may be or may not be orthogonal to the interface depending on a position where the interface intersects each scanning line L2. For example, in a case where an interface along the x direction is present so as to cross a target region, the interface is orthogonal to the beam direction around the center. However, an end of the interface is not orthogonal to the beam direction, and thus the same problem as in the first embodiment occurs.

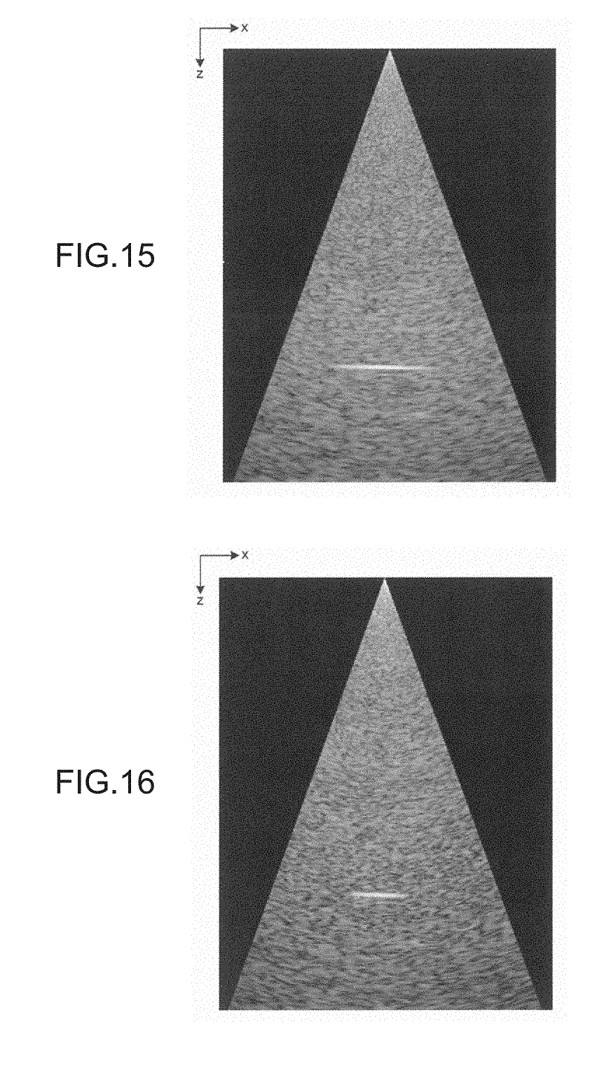

For comparative examination, as illustrated in FIG. 14, a single wire B2 was disposed along the X direction in the scanning range A2, and an ultrasonic image of the scanning range A2 in a case where ultrasonic measurement was performed was generated through simulation. FIG. 15 is a diagram illustrating an ultrasonic image generated in a case where the BF process is performed as the reception BF process, and FIG. 16 is a diagram illustrating an ultrasonic image generated in a case where the adaptive BF process is performed as the reception BF process. In this example, as illustrated in FIGS. 15 and 16, neither end of the wire B2 is depicted in generated ultrasonic images in both of cases of applying the BF process and the adaptive BF process. This is because, in the wire B2, the center vicinity is orthogonal to or substantially orthogonal to the beam direction, but an angle formed with an orthogonal direction of the beam direction increases toward the ends. A depicted range in the x direction is lower in the case of applying the adaptive BF process.

Therefore, in the second embodiment, an edge portion (beam-direction non-orthogonal line) which is not orthogonal to a beam direction is extracted for each beam direction. Regarding a process for this, first, edge detection is performed with respect to a target region, and edge portions (horizontal edges) in a horizontal direction and an inclined direction are extracted. The edge detection may be performed in the same manner as in the first embodiment. An edge portion satisfying a predetermined condition in which “the edge portion has a length of a predetermined length D_{E} set in advance or more” is extracted as a non-orthogonal line candidate.

When the respective scanning lines L2 are sequentially sampled as processing target lines, a non-orthogonal line candidate which is not orthogonal to a beam direction of a processing target line L2 is extracted as a beam-direction non-orthogonal line, and a sampling point of an intersection with the processing target line L2 is set as a BF process position. Thereafter, in the same manner as in the first embodiment, in a case where a processing target point is the BF process position, the fixed weight Wf is used as the signal combination weight W, and, in a case where a processing target point is not the BF process position, the adaptive weight Wmv is calculated to be used as the signal combination weight W.

### Functional Configuration

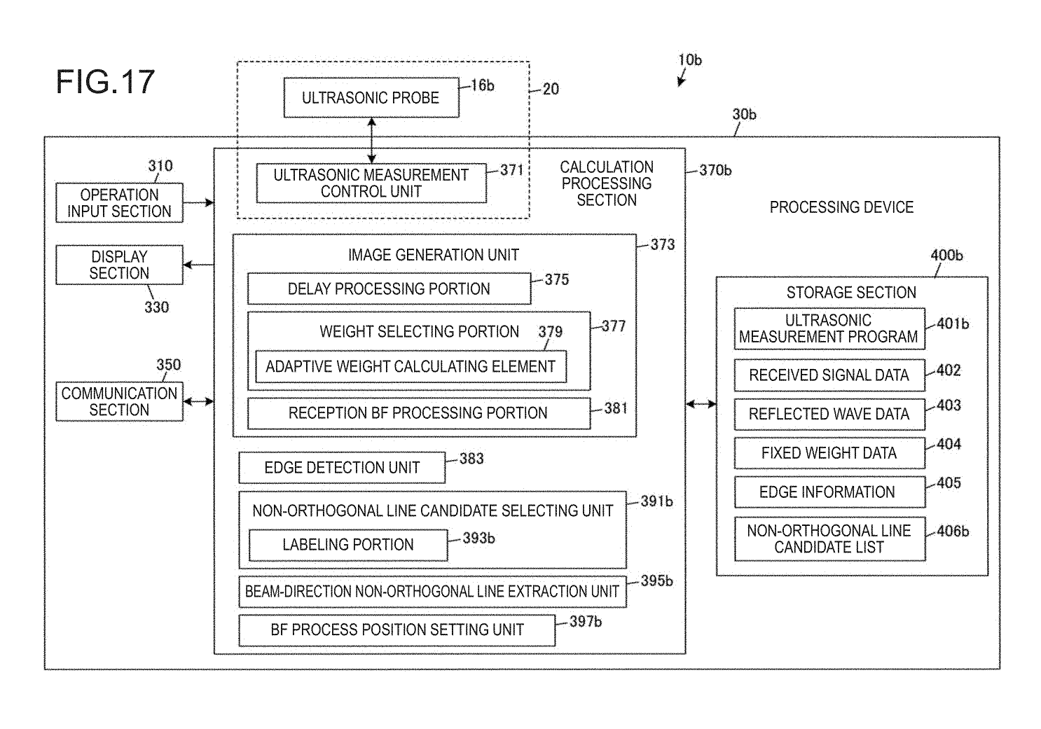

FIG. 17 is a block diagram illustrating a functional configuration example of the ultrasonic measurement apparatus 10b in the second embodiment. In FIG. 17, the ultrasonic measurement apparatus 10b includes a processing device 30b and the ultrasonic probe 16b capable of performing sector scanning. The processing device 30b includes an operation input section 310, a display section 330, a communication section 350, a calculation processing section 370b, and a storage section 400b.

In the second embodiment, the calculation processing section 370b includes an ultrasonic measurement control unit 371, an image generation unit 373, an edge detection unit 383, a non-orthogonal line candidate selecting unit 391b, a beam-direction non-orthogonal line extraction unit 395b, and a BF process position setting unit 397b.

The non-orthogonal line candidate selecting unit 391b selects an edge portion having a length of the predetermined length D_{E} or more from among edge portions detected by the edge detection unit 383 as a non-orthogonal line candidate. In this case, an edge length and an edge angle of the selected edge portion are obtained. The non-orthogonal line candidate selecting unit 391b includes a labeling portion 393b. The labeling portion 393b adds a unique label to each edge portion detected by the edge detection unit 383.

The beam-direction non-orthogonal line extraction unit 395b extracts a non-orthogonal line candidate which is not orthogonal to a beam direction of the processing target line L2 from the non-orthogonal line candidates selected by the non-orthogonal line candidate selecting unit 391b, as a beam-direction non-orthogonal line.

The BF process position setting unit 397b sets, as a BF process position, a sampling point of an intersection between the beam-direction non-orthogonal line which is extracted for the processing target line L2 by the beam-direction non-orthogonal line extraction unit 395b and the processing target line L2.

The storage section 400b stores an ultrasonic measurement program. 401b, received signal data 402, reflected wave data 403, fixed weight data 404, edge information 405, and a non-orthogonal line candidate list 406b.

The calculation processing section 370b reads and executes the ultrasonic measurement program 401b so as to realize functions of the ultrasonic measurement control unit 371, the image generation unit 373, the edge detection unit 383, the non-orthogonal line candidate selecting unit 391b, the beam-direction non-orthogonal line extraction unit 395b, the BF process position setting unit 397b, and the like. In a case where such functional units are realized by hardware such as an electronic circuit, some of programs for realizing the functions may be omitted.

An edge detection result in the edge detection unit 383 is stored in the edge information 405. In the second embodiment, the edge information 405 is rewritten by the non-orthogonal line candidate selecting unit 391b in the process of selecting a non-orthogonal line candidate.

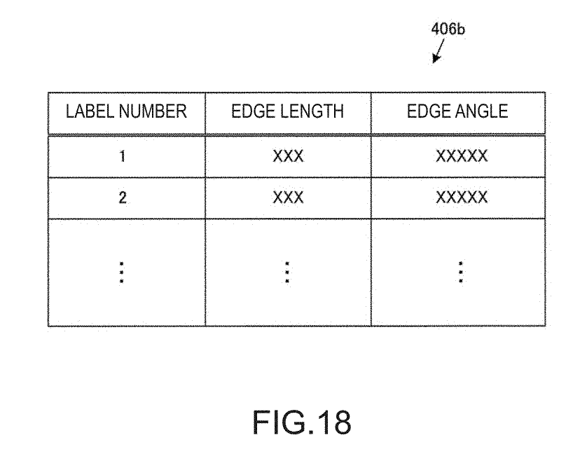

A list of edge portions selected as non-orthogonal line candidates by the non-orthogonal line candidate selecting unit 391b is stored in the non-orthogonal line candidate list 406b. FIG. 18 is a diagram illustrating a data configuration example of the non-orthogonal line candidate list 406b. As illustrated in FIG. 18, the non-orthogonal line candidate list 406b is a data table in which an edge length and an edge angle are set in correlation with a label number of a label added to an edge portion of a non-orthogonal line candidate. The non-orthogonal line candidate selecting unit 391b counts the number of sampling points forming an edge portion selected as a non-orthogonal line candidate so as to obtain the edge length, and also obtains an angle formed between a direction of the edge portion and a z direction so as to use the angle as the edge angle. The non-orthogonal line candidate selecting unit 391b generates the non-orthogonal line candidate list 406b by correlating the edge length and the edge angle with a label number.

### Flow of Process

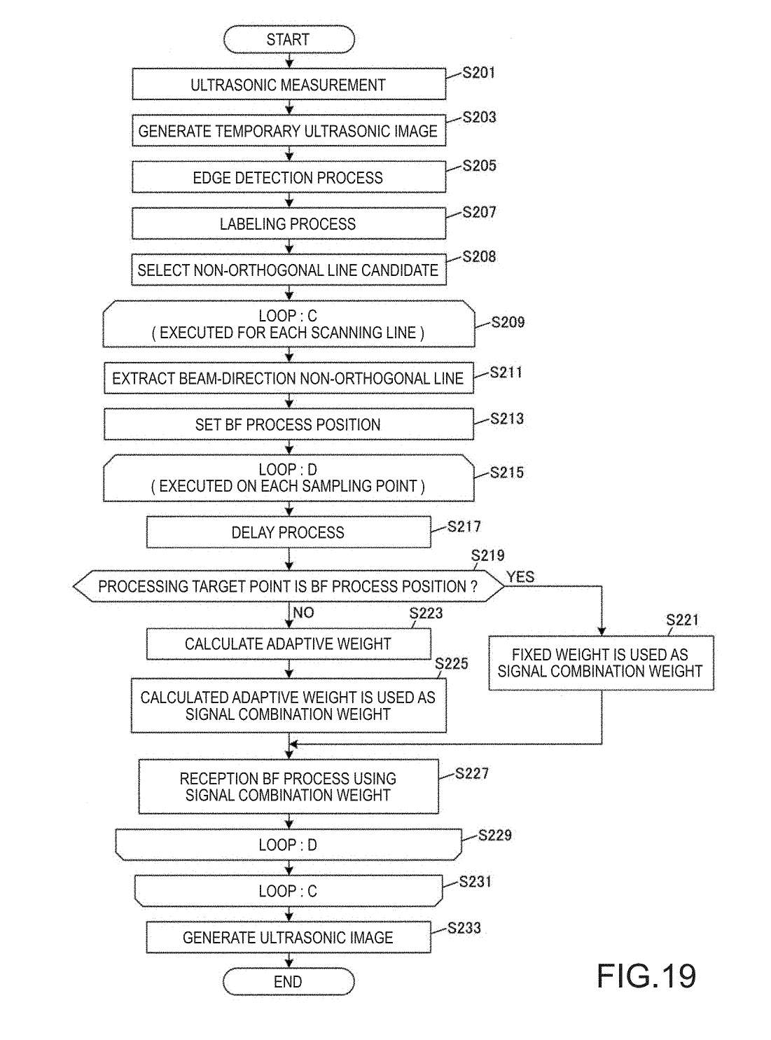

FIG. 19 is a flowchart illustrating a flow of an ultrasonic image generation process in the second embodiment. The process described here may be realized by the calculation processing section 370b reading the ultrasonic measurement program 401b from the storage section 400b and executing the ultrasonic measurement program 401b so as to operate the respective sections of the ultrasonic measurement apparatus 10b.

First, the ultrasonic measurement section 20 performs ultrasonic measurement (step S201). Next, the image generation unit 373 generates a temporary ultrasonic image (step S203).

Next, the edge detection unit 383 performs an edge detection process on the temporary ultrasonic image so as to detect edge portions present in a target region (step S205). The labeling portion 393b performs a labeling process on the edge information 405 generated through the edge detection process (step S207). Thereafter, the non-orthogonal line candidate selecting unit 391b erases an edge portion of below the predetermined length D_{E} from the edge information 405 after the labeling process, so as to select a non-orthogonal line candidate (step S208). Therefore, the non-orthogonal line candidate list 406b is generated.

In a case where the non-orthogonal line candidate is selected in the above-described way, the image generation unit 373 repeatedly performs a loop C for each scanning line L2 by referring to the received signal data 402 (step S209 to step S231). In the loop C, first, the beam-direction non-orthogonal line extraction unit 395b extracts edge portions which are not orthogonal to a beam direction of the processing target line L2 as beam-direction non-orthogonal lines among the non-orthogonal line candidates set in the non-orthogonal line candidate list 406b (step S211). Herein, for example, an angle formed between the beam direction of the processing target line L2 and the non-orthogonal line candidate is calculated by using an edge angle, and the non-orthogonal line candidate is set as a beam-direction non-orthogonal line in a case where the angle is not 90° or a predetermined range around 90° (for example, a range regarded as substantially 90°, such as 88° to 92° or 89° to 91°, which may be comprehensively said to be substantially 90° or substantially orthogonal).

Next, the BF process position setting unit 397b sets a BF process position (step S213). Herein, for example, a sampling point on the processing target line L2 added with a corresponding label in the edge information 405 is set as the BF process position on the basis of a label number of the beam-direction non-orthogonal line extracted in step S211.

In a case where the BF process position has been set, the processing target line is sampled at a predetermined time interval by using the measurement result of the ultrasonic measurement in step S201, and a process in a loop D is sequentially performed with each sampling point as a processing target point (step S215 to step S229).

In the loop D, first, the delay processing portion 375 performs a delay process of delaying a channel signal of each channel 17 by a delay time (step S217).

Next, in a case where a processing target point is set as the BF process position in step S213 (step S219: YES), the weight selecting portion 377 reads the fixed weight Wf from the fixed weight data 404 so as to use the fixed weight Wf as the signal combination weight W (step S221). In a case where a processing target point is not a BF process position (step S219: NO), the adaptive weight calculating element 379 of the weight selecting portion 377 calculates the adaptive weight Wmv according to the above-described method on the basis of a channel signal of each channel 17 after the delay process (step S223). The weight selecting portion 377 uses the calculated adaptive weight Wmv as the signal combination weight W (step S225).

Thereafter, the reception BF processing portion 381 performs the reception BF process by using the fixed weight Wf or the adaptive weight Wmv as the signal combination weight W in step S221 or step S225, and performs weighted addition on channel signals of the respective channels 17 after the delay process according to the above Equation (1) (step S227).

In a case where the process in this loop D is repeatedly performed, and sampling of the processing target line L2 is completed, the process in the loop C on the processing target line L2 is completed. In a case where the process in the loop C has been performed with all of the scanning lines L2 as processing targets, a necessary process is performed on the output z[n] from each sampling point such that an ultrasonic image is generated (step S233), and the present process is finished. The generated ultrasonic image is controlled to be displayed on the display section 330 as appropriate.

As described above, according to the second embodiment, it is possible to extract an edge portion which is not orthogonal to a beam direction and has a length of the predetermined length D_{E} or more for each beam direction. In a case where a processing target point is an edge portion extracted to correspond to a beam direction among sampling points on the processing target line L2, the reception BF process is performed by using the fixed weight Wf as the signal combination weight W, and, in a case where a processing target point is other sampling points, the reception BF process is performed by using the adaptive weight Wmv as the signal combination weight W, such that an ultrasonic image can be generated. Therefore, it is possible to achieve the same effects as in the first embodiment when an ultrasonic image is generated by scanning a target region according to a sector scanning method.

In the second embodiment, the predetermined length D_{E} is fixed, and a beam-direction non-orthogonal line is extracted. In contrast, the predetermined length D_{E} may be set to be variable by using a distance from the starting point P2 along the processing target line L2. Specifically, for example, a correspondence relationship between a distance from the starting point P2 and the predetermined length D_{E} is defined in advance. The correspondence relationship may be set as a lookup table, and may be set as a relational expression between the distance and the predetermined length D_{E}.

On the other hand, in the present modification example, all edge portions detected by the edge detection unit 383 are selected as non-orthogonal line candidates. Prior to sampling of the processing target line L2, first, all non-orthogonal line candidates which are not orthogonal to a beam direction of the processing target line L2 are selected on the basis of an edge angle. Next, a distance from the starting point P2 to an intersection between the selected non-orthogonal line candidate and the processing target line L2 is obtained, and the predetermined length D_{E} corresponding to the obtained distance is acquired on the basis of the correspondence relationship. In a case where an edge length of the selected non-orthogonal line candidate is equal to or larger than the acquired predetermined length D_{E}, the non-orthogonal line candidate is extracted as a beam-direction non-orthogonal line.

### Other Modification Examples

Ranges of values which the fixed weight Wf and the adaptive weight Wmv can take are different from each other. Thus, the fixed weight Wf may be defined depending on a value which the adaptive weight Wmv can take. For example, in the above-described embodiments, the fixed weight Wf in which Wfₘ is all “1” has been exemplified (Equation (9)). In contrast, as expressed in the following Equation (10), a coefficient k₁ corresponding to a value which the adaptive weight Wmv can take may be used, and the fixed weight Wf may be defined by multiplying the fixed weight Wfₘ of each channel 17 in Equation (9) by the coefficient k₁. Alternatively, a coefficient k₂ may be defined depending on a value which the fixed weight Wf can take. In a case where the adaptive weight Wmv has been calculated, the coefficient k₂ may be multiplied by the obtained adaptive weight Wmvₘ of each channel 17. Thus, luminance unevenness can be prevented by applying the BF process or the adaptive BF process to each sampling point.

W=k₁Wf (10)

The ultrasonic measurement apparatus of embodiments of the invention is not limited to a case where an ultrasonic image of a target region in a living body is generated by using ultrasonic measurement as in the above-described embodiments, and is also similarly applicable to a case where, for example, an ultrasonic image is generated with an internal region of a structure other than a living body as a target region, and the ultrasonic measurement apparatus is used for inspection of the structure.

The entire disclosure of Japanese Patent Application No. 2016-256451 filed Dec. 28, 2016 is expressly incorporated by reference herein.

What is claimed is:

1. An ultrasonic measurement apparatus comprising:
a probe that is provided with a plurality of channels receiving ultrasonic waves; and
a calculation processing section that performs a calculation process on a received signal which is received by each channel, performs weight selection of selecting one of a predetermined weight and an adaptive weight for performing an adaptive beam forming process as a reception beam forming process, as a signal combination weight for the reception beam forming process, for each processing target point of a target region, and generates an ultrasonic image by performing the reception beam forming process,
wherein the calculation processing section performs
edge detection with respect to the target region on the basis of the received signal,
extraction of extracting an edge portion satisfying a predetermined condition with respect to a beam direction in the reception beam forming process related to the processing target point among edge portions detected through the edge detection, and
the weight selection of selecting the predetermined weight as the signal combination weight in a case where the processing target point is the extracted edge portion.

2. The ultrasonic measurement apparatus according to claim 1,
wherein the predetermined condition includes at least a condition in which an extension direction of the edge portion is not orthogonal to the beam direction.

3. The ultrasonic measurement apparatus according to claim 1,
wherein the probe is a probe capable of performing linear scanning, and
wherein the extraction is to extract the edge portion satisfying the predetermined condition by extracting the edge portion of which the extension direction is not parallel to a scanning direction in the linear scanning.

4. The ultrasonic measurement apparatus according to claim 1,
wherein the probe is a probe capable of performing linear scanning, and
wherein the extraction includes
setting the edge portion of which an extension direction is parallel to a scanning direction in the linear scanning as an exclusion target of the extraction, and
extracting an edge portion of which an extension direction is not orthogonal to the beam direction and which has a length of a predetermined length or more from edge portions which are not the exclusion target as the edge portion satisfying the predetermined condition.

5. The ultrasonic measurement apparatus according to claim 1,
wherein the probe is a probe capable of performing sector scanning,
wherein the extraction includes extracting the edge portion satisfying the predetermined condition for each beam direction, and
wherein the weight selection includes using the predetermined weight as the signal combination weight in a case where the edge portion extracted to correspond to the beam direction is the processing target point.

6. The ultrasonic measurement apparatus according to claim 5,
wherein the predetermined condition includes a condition in which an edge portion has a length of a predetermined length or more, and
wherein the calculation processing section sets the predetermined length to be variable depending on a distance to the processing target point along the beam direction.

7. The ultrasonic measurement apparatus according to claim 1,
wherein the predetermined weight is defined depending on a value which the adaptive weight can take.

8. The ultrasonic measurement apparatus according to claim 1,
wherein the adaptive weight is a weight in which a value corresponding to the amplitude of the received signal of each channel is variable, and
wherein the adaptive beam forming process is a signal combination process in which weighted addition is performed on the received signal by using the adaptive weight.

9. A control method for an ultrasonic measurement apparatus including a probe provided with a plurality of channels receiving ultrasonic waves, the control method comprising:
performing edge detection with respect to a target region on the basis of a received signal which is received by each of the channels;
extracting an edge portion satisfying a predetermined condition with respect to a beam direction in a reception beam forming process related to a processing target point among edge portions detected through the edge detection; and
performing weight selection of selecting one of a predetermined weight and an adaptive weight for performing an adaptive beam forming process as a reception beam forming process, as a signal combination weight for the reception beam forming process, for each processing target point of the target region, and performing the reception beam forming process,
wherein the weight selection includes selecting the predetermined weight as the signal combination weight in a case where the processing target point is the extracted edge portion.
